(19) United States
(12) Patent Application Publication
(10) Pub. No.: US 2014/0276085 A1
(43) Pub. Date: Sep. 18, 2014

(54) COREGISTERED INTRAVASCULAR AND ANGIOGRAPHIC IMAGES

(71) Applicant: Volcano Corporation, San Diego, CA (US)

(72) Inventor: David G. Miller, North Andover, MA (US)

(73) Assignee: VOLCANO CORPORATION, San Diego, CA (US)

(21) Appl. No.: 14/205,806

(22) Filed: Mar. 12, 2014

Related U.S. Application Data
(60) Provisional application No. 61/779,610, filed on Mar. 13, 2013.

(51) Int. Cl. A61B 8/00

(52) U.S. Cl. CPC A61B 8/445
USPC 600/467

(57) ABSTRACT

Presenting intravascular images and angiographic images co-registered on a display. As an imaging catheter is pushed through an occlusion, a center point of the catheter and an outline of the vessel wall can be displayed. A physician can see where the catheter is crossing the occlusion within the vessel. The physician can determine what path the catheter is taking the through occlusion. Methods include extending an imaging catheter through the occlusion, determining a location of a wall of the vessel relative to a center point of the catheter, obtaining a position of the catheter within a lumen of the vessel via angiography, and displaying the catheter crossing the occlusion by co-registering the location of the wall, the position of the catheter within the lumen, and the lumen on a display.

### CROSS-REFERENCE TO RELATED APPLICATION

This application claims the benefit of, and priority to, U.S. Provisional Patent Application No. 61/779,610, filed Mar. 13, 2013, the contents of which are incorporated by reference.

### FIELD OF THE INVENTION

The invention generally relates to intravascular imaging and methods for co-registering intravascular images with angiographic images.

### BACKGROUND

People die from heart attacks. Heart attacks can be caused by the slow buildup of atherosclerotic plaque inside the blood vessels. The buildup of plaque occludes the flow of blood, and thus nutrients and oxygen, to a person's tissue and brain. Sometimes chunks of the atherosclerotic plaque break away and flow through the person's blood vessels. This can lead to serious and deadly strokes and heart attacks. If the plaque buildup is extensive enough, it will fully occlude the flow of blood, forming what is called a chronic total occlusion or CTO. If a CTO is not opened up, it can be fatal.

One approach to treating a CTO is to use an intravascular guidewire and catheter to cross the occlusion. By pushing through the occlusion, it is opened up for blood flow. With existing technology, crossing a CTO is planned by imaging the proximal and distal sections of the diseased artery by contrast angiography. The CTO is not seen because there is no blood flow to deliver the contrast dye. Crossing the CTO is done by advancing wires and catheters by tactile feel. Intravascular ultrasound imaging, IVUS, is sometimes employed, but relating the two imaging modalities with their individual displays must be imagined by the physician. The physician must also visualize the occluded vessel with his mind's eye in three dimensional space.

### SUMMARY

The invention provides systems and methods for presenting intravascular images and angiographic images co-registered on a display. As an imaging catheter is pushed through an occlusion, a center point of the catheter and an outline of the vessel wall can be displayed on a display such as an MSCT display. Thus the physician can see where the imaging catheter is crossing the occlusion and where this is within the outer wall of the vessel. The physician can determine what path the catheter is taking the through occlusion even where this could not be gleaned from only the angiography. The information can be rendered as dots and border rings on the co-registered displays. The border from the intravascular images may be determined by border detection algorithms or may be traced onto a screen with a light pen by the physician. The resulting displayed images are an improvement over the present process where the location of the vessel wall relative to the wire and catheter can only be imagined by the physician.

In certain aspects, the invention provides methods and systems for crossing a chronic total occlusion that includes extending an imaging catheter through the occlusion, determining a location of a wall of the vessel relative to a center point of the catheter, obtaining a position of the catheter within a lumen of the vessel via angiography, and displaying the catheter crossing the occlusion by co-registering the location of the wall, the position of the catheter within the lumen, and the lumen on a display. In some embodiments, the detected border is detected by multi-slice computed tomography. Optionally, a vessel outline is generated by multi-slice computed tomography. In some embodiments, the display comprises at least one center dot showing the center point of the catheter and at least one border ring showing the location of the wall.

In some embodiments, the imaging catheter is an IVUS catheter. The method may further include matching a series of B-scans to an outline of the vessel. A border may be detected via a border detection algorithm.

Determining the location of the wall of the vessel may be done by performing an intravascular imaging operation to obtain intravascular image data. The intravascular image data may be transformed to represent a rotation of an intravascular image. This rotation can provide a best fit between the determined location and a detected border.

In some embodiments, a tip of the imaging catheter is re-oriented and the catheter is used to capture second image data. The method may further include performing an alignment using the intravascular image data and the second image data.

In certain embodiments, the method includes advancing the imaging catheter and obtaining a series of angiographic images. A distance that the imaging catheter advances can be measured using a caliper.

The method may further include doing an IVUS pullback to obtain a vessel wall.

A position of the imaging catheter may be sensed by triangulation (e.g., using low frequency ultrasound transducers electromagnets, or light sources and a light sensor on the catheter).

### BRIEF DESCRIPTION OF THE DRAWINGS

FIG. 1 shows an intravascular imaging system.

FIG. 2 gives a diagram of components of an IVUS system.

FIG. 3 shows an IVUS control station.

FIG. 4 is a schematic of components within an IVUS system.

FIG. 5 shows a caliper.

FIG. 6 is a diagram of components of an OCT system.

FIG. 7 gives a detailed view of an OCT imaging engine.

FIG. 8 is a schematic of an OCT patient interface module.

FIG. 9 shows a pattern that an OCT imaging fiber traces during a pullback.

FIG. 10 diagrams a pattern of scan lines produced by an imaging operation.

FIG. 11 is a reproduction of a display from an intravascular imaging system.

FIG. 12 is an illustration of a display from an intravascular imaging system.

### DETAILED DESCRIPTION

The invention provides systems and methods for coordinating operations during intravascular imaging. Any intravascular imaging system may be used in systems and methods of the invention. Systems and methods of the invention have application in intravascular imaging methodologies such as intravascular ultrasound (IVUS) and optical coherence tomography (OCT) among others that produce a three-dimensional image of a vessel.

The invention provides system and methods for displaying co-registered images of arteries including a chronic total occlusion (CTO). One or more angiographic images are co-registered with images from MSCT systems, IVUS systems, or both. The B-scan IVUS display with proper processing is able to provide two pieces of useful information, the center of the IVUS catheter and the outline of the vessel border. Multi-slice computed tomography, MSCT, is able to create surface rendered images of the vessel proximal to the CTO. This provides detailed information on the size and location of the vessel wall. MSCT equipment acquires a vast amount of data that is used to generate a variety of views on the displays. Conventional angiographic images taken at the time of the CTO crossing reveal the IVUS catheters position in the vessel relative to the CTO. As described below, this permits a series of IVUS B-scans to be matched to the outline of the vessel generated by the MSCT. The IVUS image information and the angiographic information is co-registered on the MSCT displays using image co-registration algorithms. As the catheter is pushed through the CTO the center point of the catheter and the outline of the vessel wall can be periodically displayed on the MSCT displays. From the center point and vessel wall data from the IVUS image periodically displayed, the path that the catheter is taking can be judged where the vessel cannot be seen by angiography. This imaging information is presented as a series of center dots and a border rings drawn on the co-registered displays. The border from the IVUS images may be determined by border detection algorithms or may be traced onto a screen with a light pen by the physician. Border detection is described in U.S. Provisional Application No. 61/739,920, filed Dec. 20, 2012, incorporated by reference. The resulting displayed images are an improvement over the present process where the location of the vessel wall relative to the wire and catheter can only be imagined by the physician.

Image co-registration software provides the capability to combine MSCT, angiography, IVUS and displacement information on one display with multiple views or displays with multiple views of the three dimensional volume around the physiology of interest. Co-registration software rotates the IVUS image to best fit the border derived from the MSCT data. If the position of the catheter is ambiguous the catheter tip is deflected or moved laterally and another pair of images are recorded. This guarantees an eccentricity of the vessel wall and a second image that permits a solution to the alignment function. The axial placement of the IVUS image inside the vessel is known because it is against the CTO. In cases of a CTO at a side branch of the vessel a button on the console is pushed to indicate the initial position of the IVUS catheter in the vessel.

Another source of imaging data is magnetic resonance imaging, MRI. In some cases this is also a source of input data and is used in the same manner that the MSCT data is used. This is detailed image data that is acquired in advance of the CTO crossing procedure.

Methods include acquiring an MSCT or IVUS image of an artery using a three-dimensional medical imaging device, wherein the MSCT or IVUS image includes imagery of the CTO. An angiographic image is also obtained. The MSCT or IVUS image is co-registered with the angiographic image data using an image processing device. The co-registered image data are displayed in on a display device to show the CTO.

Co-registering angiography with MSCT or IVUS images may include segmenting the three-dimensional image data. The displayed co-registered image data may be used for guidance in performing percutaneous coronary intervention (PCI) for coronary arteries.

In some embodiment, initial images are acquired. The initial images may be three-dimensional CT image data, for example, multi-slice computed tomography (MSCT) image data. MSCT is an example of a CT modality that can capture fine structural details of the subject anatomy. For example, using this modality, individual vessels may be clearly imaged and plaque lining the vessels may be identified. See, e.g., U.S. Pub. 2010/0061611 to Xu, the contents of which are incorporated by reference. MSCT image slices may show occluded coronary arteries.

The initial images may be four-dimensional MSCT image data. Four-dimensional MSCT image data may capture imagery showing the three spatial dimensions as well as time. In this way, four-dimensional MSCT image data captures motion. Electrocardiography (ECG) data may be recorded along with the four-dimensional MSCT image data so that the progression of motion may be indexed to the stages of the cardiac cycle so that the full range of motion of the heart and coronary arteries may be understood. The initial images may also or alternatively include magnetic resonance imagery (MRI) data that may be co-registered to the fluoroscope image sequence.

After the initial images are acquired, radio-contrast may be administered and angiographic images may be taken. Angiography may be performed, for example, using one or more fluoroscopes, each mounted on a c-arm. Where multiple fluoroscopes are used, for example, to achieve higher accuracy and/or to further constrain co-registration, each may be positioned at a unique angle. The angle between the two fluoroscopic sequences may be between 30 degrees and 90 degrees. The fluoroscope image sequence(s) may be two-dimensional.

FIG. 1 depicts an exemplary layout of an intravascular imaging system 101 as may be found, for example, in a catheter lab. An operator uses control station 110 and navigational device 125 to operate catheter 112 via patient interface module (PIM) 105. At a distal tip of catheter 112 is imaging tip 114. Computer device 120 works with PIM 105 to coordinate imaging operations. Imaging operations proceed by using catheter 112 to image the patient's tissue. The image data is received by device 120 and interpreted to provide an image on monitor 103. System 101 is operable for use during diagnostic imaging of the peripheral and coronary vasculature of the patient. System 101 can be configured to automatically visualize boundary features, perform spectral analysis of vascular features, provide qualitative or quantitate blood flow data, or a combination thereof.

In some embodiments, operation of system 101 employs a sterile, single use intravascular ultrasound imaging catheter 112. Catheter 112 is inserted into the coronary arteries and vessels of the peripheral vasculature under the guidance of angiogrpahic system 107. System 101 may be integrated into existing and newly installed catheter laboratories (angiography suites.) The system configuration is flexible in order to fit into the existing catheter laboratory work flow and environment. For example, the system can include industry standard input/output interfaces for hardware such as navigation device 125, which can be a bedside mounted joystick. System 101 can include interfaces for one or more of an EKG system, exam room monitor, bedside rail mounted monitor, ceiling mounted exam room monitor, and server room computer hardware.

System 101 connects to catheter 112 via PIM 105, which may contain a type CF (intended for direct cardiac application) defibrillator proof isolation boundary. All other input/output interfaces within the patient environment may utilize both primary and secondary protective earth connections to limit enclosure leakage currents. The primary protective earth connection for controller 125 and control station 110 can be provided through the bedside rail mount. A secondary connection may be via a safety ground wire directly to the bedside protective earth system. Monitor 103 and an EKG interface can utilize the existing protective earth connections of the monitor and EKG system and a secondary protective earth connection from the bedside protective earth bus to the main chassis potential equalization post.

Computer device 120 can include a high performance dual Xeon based system using an operating system such as Windows XP professional. Computer device 120 may be configured to perform real time intravascular ultrasound imaging while simultaneously running a tissue classification algorithm referred to as virtual histology (VH). The application software can include a DICOM3 compliant interface, a work list client interface, interfaces for connection to angiographic systems, or a combination thereof. Computer device 120 may be located in a separate control room, the exam room, or in an equipment room and may be coupled to one or more of a custom control station, a second control station, a joystick controller, a PS2 keyboard with touchpad, a mouse, or any other computer control device.

Computer device 120 may generally include one or more USB or similar interfaces for connecting peripheral equipment. Available USB devices for connection include the custom control stations, the joystick, and a color printer. In some embodiments, control system includes one or more of a USB 2.0 high speed interface, a 50/100/1000 baseT Ethernet network interface, AC power input, PS2 jack, potential equalization post, 1 GigE Ethernet interface, microphone & line inputs, line output VGA Video, DVI video interface, PIM interface, ECG interface, other connections, or a combination thereof. As shown in FIG. 1, computer device 120 is generally linked to control station 110.

Control station 110 may be provided by any suitable device, such as a computer terminal (e.g., on a kiosk). In some embodiments, control system 110 is a purpose built device with a custom form factor (e.g., as shown in FIG. 12).

In certain embodiments, systems and methods of the invention include processing hardware configured to interact with more than one different three dimensional imaging system so that the tissue imaging devices and methods described here in can be alternatively used with OCT, IVUS, or other hardware.

Any target can be imaged by methods and systems of the invention including, for example, bodily tissue. In certain embodiments, systems and methods of the invention image within a lumen of tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

The invention provides methods of co-registering angiographic images with IVUS images. An angiography system may be used with an intravascular imaging system (e.g., OCT, IVUS, or optical-acoustic imaging). Angiography systems can be used to visualize the blood vessels by injecting a radio-opaque contrast agent into the blood vessel and imaging using X-ray based techniques such as fluoroscopy. Angiographic techniques include projection radiography as well as imaging techniques such as CT angiography and MR angiography. In certain embodiments, angiography involves using a catheter to administer the x-ray contrast agent at the desired area to be visualized. The catheter is threaded into an artery, and the tip is advanced through the arterial system into the major coronary artery. X-ray images of the transient radio contrast distribution within the blood flowing within the coronary arteries allows visualization of the size of the artery openings. Features and media within the blood and walls of the arteries are studied. Angiography systems and methods are discussed, for example, in U.S. Pat. No. 7,734,009; U.S. Pat. No. 7,564,949; U.S. Pat. No. 6,520,677; U.S. Pat. No. 5,848,121; U.S. Pat. No. 5,346,689; U.S. Pat. No. 5,266,302; U.S. Pat. No. 4,432,370; and U.S. Pub. 2011/0301684, the contents of each of which are incorporated by reference in their entirety for all purposes.

The angiography system can be used to detect a change. The angiography system can be used to detect the flush with saline (e.g., the temporary displacement of the radiopaque dye by the saline), the initial influx of radiopaque dye, or other such flushes. A processor that receives the angiography signal data can detect a brightness or contrast change (e.g., by digital signal processing techniques including those described in Smith, 1997, THE SCIENTIST AND ENGINEERS GUIDE TO DIGITAL SIGNAL PROCESSING, California Technical Publishing (San Diego, Calif.) 626 pages, the contents of which are hereby incorporated by reference).

The angiography images will be co-registered to one or more images from an IVUS system or the like.

IVUS uses a catheter with an ultrasound probe attached at the distal end. The proximal end of the catheter is attached to computerized ultrasound equipment. To visualize a vessel via IVUS, angiographic techniques are used and the physician positions the tip of a guide wire, usually 0.36 mm (0.014″) diameter and about 200 cm long. The physician steers the guide wire from outside the body, through angiography catheters and into the blood vessel branch to be imaged.

The ultrasound catheter tip is slid in over the guide wire and positioned, again, using angiography techniques, so that the tip is at the farthest away position to be imaged. Sound waves are emitted from the catheter tip (e.g., in about a 20-40 MHz range) and the catheter also receives and conducts the return echo information out to the external computerized ultrasound equipment, which constructs and displays a real time ultrasound image of a thin section of the blood vessel currently surrounding the catheter tip, usually displayed at 30 frames/second image.

The guide wire is kept stationary and the ultrasound catheter tip is slid backwards, usually under motorized control at a pullback speed of 0.5 mm/s. Systems for IVUS are discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety. Imaging tissue by IVUS produces tomographic (cross-sectional) or ILD images, for example, as shown in FIG. 10 and illustrated in FIG. 11. An IVUS system can be installed substantially as shown in FIG. 1. An IVUS computer device 120 takes the place of OCT computer device 120.

FIG. 2 describes an exemplary computer device 120 according to certain embodiments. Computer device 120 may include a motherboard 529 that includes an IVUS signal generation and processing system. The signal generation and processing system may comprises an analog printed circuit assembly (PCA) 531, an digital PCA 533, one or more filter modules, and a VH board 535. Analog PCA 531 and digital PCA 533 are used to excite transducer 514 via catheter 512 and to receive and process the gray scale IVUS signals. The VH board 535 is used to capture and pre-process the IVUS RF signals and transfer them to the main VH processing algorithm as run by a computer processor system (e.g., dual Xeon processors). PIM 105 is directly connected to the analog PCA 531.

FIG. 3 shows a control station 110 according to certain embodiments. A slide out keyboard is located on the bottom for manual text entry. Control station 110 may be designed for different installations options. The station can be placed directly on a desktop surface. With an optional bedside mounting kit, control station 110 can be affixed directly to the bedside rail. Control station 110 can include a standard four hole VESA mount on the underside to allow other mounting configurations. Control system 110 may provide a simple-to-use interface with frequently-operated functions mapped to unique switches. Control station 110 may be powered from, and may communicate with, computer 120 using a standard USB 1.1 interface. The system may include a control panel 515. In some embodiments, multiple control panels 515 are mounted in both the exam room and/or the control room. Control system 110 can have a surface control panel with buttons for frequently-operated functions (e.g., as contact closure switches). Those dome switches are covered with a membrane overlay. The use of dome switches provides a tactile feedback to the operator upon closure. The control panel may include a pointing device such as a trackball to navigate a pointer on the graphical user interface of the system. The control panel may include several screen selection keys. The settings key is used to change system settings like date and time and also permits setting and editing default configurations. The display key may be used to provide enlarged view for printing. In some embodiments, the print key prints a 6×4 inch photo of the current image on the screen. The control panel may include a Ring Down key that toggles the operation of ringdown subtraction. A chroma key can turn blood flow operations on and off. The VH key can operate the virtual histology engine. A record, stop, play, and save frame key are included for video operation. Typically, the home key will operate to display the live image. A menu key provides access to measurement options such as diameter, length, and borders. Bookmark can be used while recording a loop to select specific areas of interest. Select (+) and Menu (−) keys are used to make selections.

In some embodiments, the system includes a joystick for navigational device 525. The joystick may be a sealed off-the-shelf USB pointing device used to move the cursor on the graphical user interface from the bedside. System 501 may include a control room monitor, e.g., an off-the-shelf 59″ flat panel monitor with a native pixel resolution of 5280×1024 to accept DVI-D, DVI-I and VGA video inputs.

As shown in FIG. 1, control station 110 is operably coupled to PIM 105, from which catheter 112 extends. Catheter 112 includes an ultrasound transducer 114 located at the tip. Any suitable IVUS transducer may be used. For example, in some embodiments, transducer 114 is driven as a synthetic aperture imaging element. Imaging transducer 114 may be approximately 5 mm in diameter and 2.5 mm in length. In certain embodiments, transducer 114 includes a piezoelectric component such as, for example, lead zirconium nitrate or PZT ceramic. The transducer may be provided as an array of elements (e.g., 64), for example, bonded to a Kapton flexible circuit board providing one or more integrated circuits. This printed circuit assembly may rolled around a central metal tube, back filled with an acoustic backing material and bonded to the tip of catheter 112. In some embodiments, signals are passed to the system via a plurality of wires (e.g., 7) that run the full length of catheter 112. The wires are bonded to the transducer flex circuit at one end and to a mating connector in PIM 105 at the other. The PIM connector may also contains a configuration EPROM. The EPROM may contain the catheter's model and serial numbers and the calibration coefficients which are used by the system. The PIM 105 provides the patient electrical isolation, the beam steering, and the RF amplification. PIM 105 may additionally include a local microcontroller to monitor the performance of the system and reset the PIM to a known safe state in the event of loss of communication or system failure. PIM 105 may communicate with computer device 120 via a low speed RS232 serial link.

FIG. 4 provides a schematic of analog PCA 531 and digital PCA 533 according to certain embodiments of the invention. Analog PCA 531 is shown to include amplifier 541, band pass filter 545, mixer 549, low pass filter 553, and analog-to-digital converter (ADC) 157. (Here, the system is depicted as being operable to convert the transducer RF data to “In-Phase” and “Quadrature” (IQ) data. According to this embodiment, ADC 157 is 52-bits wide and converts the IQ data to a dual digital data stream.) Analog board 531 further includes an interface module 561 for PIM 105, as well as a clock device 569.

Digital PCA 533 is depicted as having an acquisition FPGA 165, as well as a focus FPGA 171, and a scan conversion FPGA 179. Focus FPGA 171 provides the synthetic aperture signal processing and scan conversion FPGA 179 provides the final scan conversion of the transducer vector data to Cartesian coordinates suitable for display via a standard computer graphics card on monitor 503. Digital board 533 further optionally includes a safety microcontroller 581, operable to shut down PIM 105 as a failsafe mechanism. Preferably, digital PCA 533 further includes a PCI interface chip 575. It will be appreciated that this provides but one exemplary illustrative embodiment and that one or skill in the art will recognize that variant and alternative arrangements may perform the functions described herein. Clock device 569 and acquisition FPGA 165 operate in synchronization to control the transmission of acquisition sequences. FIG. 4 presents one exemplary system architecture, and other IVUS systems are known in the art and may be used for flush-triggered IVUS imaging. For flush-triggered imaging, the blood is displaced by a solution, and this flushing step is detected. Any suitable detection mechanism can be used including, for example, blood pressure or angio systems as discussed above.

In some embodiments, an initial co-registration of angiographic to other images is performed. The initial co-registration procedure may match the fluoroscope image sequence with the MSCT image data by identifying an ECG phase of the MSCT data and then selecting a frame from the fluoroscope sequence that has the same ECG phase. See, e.g., U.S. Pub. 2010/0061611 to Xu. A rough alignment may then be performed, for example, using DICOM information from the MSCT and C-arm geometry from typically one or two fluoroscopic sequences. When two fluoroscopic sequences are used to achieve higher accuracy, proper breathing compensation may be used to provide for valid reconstructed 3D landmark points and a valid registration result.

After initial registration, breathing motion compensation may be achieved by tracking the guidewire throughout the execution of the intervention procedure and the registration may be updated locally to follow a motion estimated from guidewire tracking by applying co-registration between the MSCT coronary centerline and tracked guidewire result.

After the initial co-registration, a registration procedure may then be employed. Any co-registration procedure may be used. The invention includes methods and systems for co-registration.

In some embodiments, as the IVUS catheter is advanced a series of angiographic images are periodically obtained. These are co-registered and permit the vessel borders and the center of the IVUS catheter to be placed on the displays in the proper locations.

In certain embodiments, the IVUS catheter is equipped with a digital caliper that measures the distance that the IVUS catheter advances in the guide catheter and provides the displacement information to the system.

FIG. 5. depicts a caliper 301 for use with the invention. Caliper 301 houses in a housing 317 a catheter 337 and optionally a guide catheter 305. Catheter 337 extends from an end that includes a hemostatic valve 333 and a magnetic contact 325, extending from a sliding caliper 321. The other end can bear a luer lock nut 309, a connection for electrical cable 341, or both.

The caliper is attached to the guide catheter which is taped to the patient's body to fix its location. Hemostatic valve 333 fixes the slide of the caliper to the IVUS catheter.

The IVUS catheter is placed against the CTO and an angiographic and an IVUS image is recorded. The distance measurement on caliper 301 is recorded.

In some embodiments, IVUS and angiography is used without the MSCT information. The 3D vessel wall is established by doing an IVUS pull back. A pull back will require a simple readjustment of caliper 301 to set slide 321 to the other end of its travel range. After the pull back the slide is set at the other end of its travel to enable forward motion into the CTO.

In certain embodiments, several low frequency ultrasound transducers are placed on the patient's body to allow the sensing of the catheter to identify the location of the catheter tip in three dimensional space by triangulation. The low frequency of the surface transducers corresponds to the lateral mode frequency of the US transducer on the IVUS catheter. The locations of the transmitters on the body are visible with radiography to permit the co-registration on the angiography displays.

In some embodiments, a plurality of pulsed electromagnets are placed on the patient's body and driven with a coded signal to allow the sensing of the catheter to identify the location of the catheter tip in three dimensional space by triangulation. The catheter has coils incorporated in the tip to sense the strength of the magnetic field at the catheter. The locations of the electromagnets on the body are visible with angiography to permit the co-registration on the angiography displays.

In certain embodiments, several pulsed infrared or visible light sources are placed on the patient's body and driven with a coded signal to allow the photo detector on the catheter to identify the location of the catheter tip in three dimensional space by triangulation. The catheter has light sensor in the tip to sense the time of flight of the magnetic field to the catheter. The locations of the light sources on the body are visible with radiography to permit the co-registration on the angiography displays.

One of ordinary skill in the art will recognize that one or a combination of the foregoing embodiments may be used to co-register the images. In some embodiments, a system for displaying co-registered images includes an angiographic subsystem and an IVUS subsystem (and optionally an MSCT subsystem). An image processing device co-registers acquired images. Monitor 103 may display the co-registered images.

The image processing device may execute a co-registration routine to perform method steps including segmenting the three-dimensional image data; identifying a vessel structure within the segmented image data by detecting a centerline path; determining an optimal articulation of the one or more fluoroscopes and setting each of the one or more fluoroscopes to the respective optimal articulation while real-time image data is acquired; performing an initial co-registration of coronary arteries using the identified vessel structure within the three-dimensional image data and the real-time image data; automatically estimating a registration matrix for distorting the three-dimensional image data to continuously align with the real-time image data based on the initial co-registration; and rendering a superimposed visualization by combining the three-dimensional image data and the real-time image data according to the estimated registration matrix. A computer system includes a processor coupled to a tangible, non-transitory memory operable to cause the system to perform the method steps.

While discussed above in terms of IVUS, it is recognized that co-registration according to methods herein by operate to co-register angiographic images with OCT images.

In an exemplary embodiment, the invention provides a system for capturing a three dimensional image by OCT. Commercially available OCT systems are employed in diverse applications such as art conservation and diagnostic medicine, e.g., ophthalmology. OCT is also used in interventional cardiology, for example, to help diagnose coronary artery disease. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety.

In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path interferometers are further described for example in U.S. Pat. No. 7,999,938; U.S. Pat. No. 7,995,210; and U.S. Pat. No. 7,787,127, the contents of each of which are incorporated by reference herein in its entirety.

In a differential beam path system, amplified light from a light source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in U.S. Pat. No. 7,783,337; U.S. Pat. No. 6,134,003; and U.S. Pat. No. 6,421,164, the contents of each of which are incorporated by reference herein in its entirety.

FIG. 6 presents a high-level diagram of a differential beam path OCT system according to certain embodiments of the invention. For intravascular imaging, a light beam is delivered to the vessel lumen via a fiber-optic based imaging catheter 826. The imaging catheter is connected through hardware to software on a host workstation. The hardware includes an imagining engine 859 and a handheld patient interface module (OCT PIM) 839 that includes user controls. The proximal end of the imaging catheter is connected to OCT PIM 839, which is connected to an imaging engine as shown in FIG. 7.

FIG. 7 gives a detailed view of components of imaging engine 859 (e.g., a bedside unit). Imaging engine 859 houses a power supply 849, light source 827, interferometer 931, and variable delay line 835 as well as a data acquisition (DAQ) board 855 and optical controller board (OCB) 854. A PIM cable 841 connects the imagine engine 859 to the OCT PIM 839 and an engine cable 845 connects the imaging engine 859 to the host workstation.

FIG. 8 shows components of OCT PIM 839. PIM 839 includes a pullback motor 865 and a spin motor 861 to displace and rotate the OCT imaging element via a proximal end mounted to the pullback carriage while a distal end is inside of a lumen of a vessel. Light is transmitted as a sample path from the imaging element into the patient's tissue, from where it is reflected back.

Within the imaging engine, light for image capture originates within a light source. This light is split between an OCT interferometer and an auxiliary, or “clock”, interferometer. Light directed to the OCT interferometer is further split by a splitter and recombined by another splitter with an asymmetric split ratio. The majority of the light is guided into a sample path and the remainder into a reference path. The sample path includes optical fibers running through the OCT PIM 839 and the imaging catheter 826 and terminating at the distal end of the imaging catheter where the image is captured.

Typical intravascular OCT involves introducing the imaging catheter into a patient's target vessel using standard interventional techniques and tools such as a guide wire, guide catheter, and angiography system. Rotation is driven by spin motor 861 while translation is driven by pullback motor 865.

FIG. 9 describes the motion for image capture defined by rotation and translation. Blood in the vessel is temporarily flushed with a clear solution for imaging. Using light provided by the imaging engine, the inner core sends light into the tissue in an array of A scan lines as illustrated in FIG. 10 and detects reflected light.

FIG. 10 shows the positioning of A scans with in a vessel. Each place where one of A scans A11, A12, . . . , AN intersects a surface of a feature within vessel 101 (e.g., a vessel wall) coherent light is reflected and detected. Catheter 826 translates along axis 117 being pushed or pulled by pullback motor 865.

The reflected, detected light is transmitted along a sample path of interferometer 831 to be recombined with the light from reference path via a splitter. A variable delay line (VDL) 925 on the reference path uses an adjustable fiber coil to match the length of reference path to the length of sample path. The reference path length may adjusted by a stepper motor translating a mirror on a translation stage under the control of firmware or software. The free-space optical beam on the inside of the VDL 925 experiences more delay as the mirror moves away from the fixed input/output fiber.

The combined light from the splitter is split into orthogonal polarization states, resulting in RF-band polarization-diverse temporal interference fringe signals. The interference fringe signals are converted to photocurrents using PIN photodiodes on the OCB 851 as shown in FIG. 7. The interfering, polarization splitting, and detection steps are done by a polarization diversity module (PDM) on the OCB. Signal from the OCB is sent to the DAQ 855, shown in FIG. 7. The DAQ includes a digital signal processing (DSP) microprocessor and a field programmable gate array (FPGA) to digitize signals and communicate with the host workstation and the PIM. The FPGA converts raw optical interference signals into meaningful OCT images. The DAQ also compresses data as necessary to reduce image transfer bandwidth to 1 Gbps (e.g., compressing frames with a lossy compression JPEG encoder).

Data is collected from A scans A11, A12, . . . , AN and stored in a tangible, non-transitory memory. A set of A scans generally define a B scan. The data of all the A scan lines together represent a three-dimensional image of the tissue. The data of the A scan lines generally referred to as a B scan can be used to create an image of a cross section of the tissue, sometimes referred to as a tomographic view. The data of the A scan lines is processed according to systems and methods of the inventions to generate images of the tissue. By processing the data appropriately (e.g., by fast Fourier transformation), a two-dimensional image can be prepared from the three dimensional data set. Systems and methods of the invention provide one or more of a tomographic view, ILD, or both.

FIG. 11 shows a display 237 including a tomographic view in the left panel. A tomographic view can be represented as a visual depiction of a cross section of a vessel (see left side of FIG. 11). Where a tomographic view generally represents an image as a planar view across a vessel or other tissue (i.e., normal to axis 117), an image can also be represented as a planar view along a vessel (i.e., axis 117 lies in the plane of the view).

FIG. 11 shows a longitudinal planar view of the vessel in the right panel. Such a planar image along a vessel is sometimes referred to as an in-line digital view or image longitudinal display (ILD). The system captures a 3D data set that is used to present the image of tissue. An electronic apparatus within the system (e.g., PC, dedicated hardware, or firmware) stores the three dimensional data set in a tangible, non-transitory memory and renders a display 237 (e.g., on a screen or computer monitor) that includes a 2D image of the tissue.

FIG. 12 shows a display similar to that shown in FIG. 11, rendered in a simplified style of the purposes of ease of understanding. Display 237 may be rendered within a windows-based operating system environment, such as Windows, Mac OS, or Linux or within a display or GUI of a specialized system. Display 237 can include any standard controls associated with a display (e.g., within a windowing environment) including minimize and close buttons, scroll bars, menus, and window resizing controls. Elements of display 237 can be provided by an operating system, windows environment, application programing interface (API), web browser, program, or combination thereof (for example, in some embodiments a computer includes an operating system in which an independent program such as a web browser runs and the independent program supplies one or more of an API to render elements of a GUI). Display 237 can further include any controls or information related to viewing images (e.g., zoom, color controls, brightness/contrast) or handling files comprising three-dimensional image data (e.g., open, save, close, select, cut, delete, etc.). Further, display 237 can include controls (e.g., buttons, sliders, tabs, switches) related to operating a three dimensional image capture system (e.g., go, stop, pause, power up, power down). As shown in FIG. 12, display 237 includes two images of tissue, a tomographic view and an ILD. As discussed above, intravascular imaging provides a very good display when blood is flushed from the vessel. The clarity of display 237 as shown in FIG. 11 and drawn in FIG. 12 relates to the ability of the imaging modality to see through the surrounding media and to the affected tissue. In high frequency IVUS, discussed in greater detail below, the imaging involves ultrasonic signals that must penetrate through the media. In OCT, the imaging involves light signals. So that the imaging signal can propagate most directly to the tissue and back, a flush operation replaces the blood with a solution that is transparent to the imaging signal (e.g., saline). Thus, the medium surrounding the image capture device does not interfere with the imaging operation. Additionally, so that image capture is well synchronized and a useful data set can be captured on every operation, the flush operation is used as the direct trigger of the image capture operation.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

Aspects of the invention provide systems operable to present intravascular images and angiographic images co-registered on a display. As an imaging catheter is pushed through an occlusion, a center point of the catheter and an outline of the vessel wall can be displayed. A computer system comprising a processor coupled to a non-transitory memory can perform data capture, co-registration, and display steps. A physician can see where the catheter is crossing the occlusion within the vessel. The physician can determine what path the catheter is taking the through occlusion. Methods include extending an imaging catheter through the occlusion and using one or more computer processors for determining a location of a wall of the vessel relative to a center point of the catheter, obtaining a position of the catheter within a lumen of the vessel via angiography, and displaying the catheter crossing the occlusion by co-registering the location of the wall, the position of the catheter within the lumen, and the lumen on a display.

Steps of the invention may be performed using systems that include dedicated medical imaging hardware, general purpose computers, or both. As one skilled in the art would recognize as necessary or best-suited for performance of the methods of the invention, computer systems or machines of the invention include one or more processors (e.g., a central processing unit (CPU) a graphics processing unit (GPU) or both), a main memory and a static memory, which communicate with each other via a bus. A computer device generally includes memory coupled to a processor and one or more input/output devices.

A processor generally includes one or more single or multi-core processors, e.g., silicon chips, such as those made by Intel (Santa Clara, Calif.).

Memory according to the invention can include a machine-readable medium on which is stored one or more sets of instructions (e.g., software), data, or both embodying any one or more of the methodologies or functions described herein. The software may also reside, completely or at least partially, within the main memory, processor, or both during execution thereof by the computer system, the main memory and the processor also constituting machine-readable media. The software may further be transmitted or received over a network via the network interface device.

Exemplary input/output devices include a monitor, an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, a medical imaging device such as an intravascular imaging catheter, an angiographic device such as an MSCT instrument, and a network interface device, which can be, for example, a network interface card (NIC), Wi-Fi card, or cellular modem.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any of the methodologies of the present invention. The term “machine-readable medium” shall accordingly be taken to include, but not be limited to, solid-state memories (e.g., subscriber identity module (SIM) card, secure digital card (SD card), micro SD card, or solid-state drive (SSD)), optical and magnetic media, and any other tangible storage media. Preferably, computer memory is a tangible, non-transitory medium, such as any of the foregoing, and may be operably coupled to a processor by a bus. Methods of the invention include writing data to memory—i.e., physically transforming arrangements of particles in computer memory so that the transformed tangible medium represents the tangible physical objects—e.g., the arterial plaque in a patient's vessel.

As used herein, the word “or” means “and or or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for crossing a chronic total occlusion, the method comprising:
extending an imaging catheter through the occlusion;
determining a location of a wall of the vessel relative to a center point of the catheter;
obtaining a position of the catheter within a lumen of the vessel via angiography; and
displaying the catheter crossing the occlusion by co-registering the location of the wall, the position of the catheter within the lumen, and the lumen on a display.

2. The method of claim 1, wherein the display comprises at least one center dot showing the center point of the catheter and at least one border ring showing the location of the wall.

3. The method of claim 1, further comprising generating a vessel outline by multi-slice computed tomography.

4. The method of claim 1, wherein the imaging catheter is an IVUS catheter.

5. The method of claim 1, further comprising matching a series of B-scans to an outline of the vessel.

6. The method of claim 1, further comprising detecting a border via a border detection algorithm.

7. The method of claim 1, wherein determining the location of the wall of the vessel comprises performing an intravascular imaging operation to obtain intravascular image data.

8. The method of claim 7, further comprising transforming the intravascular image data to represent a rotation of an intravascular image.

9. The method of claim 8, wherein the rotation provides a best fit between the determined location and a detected border.

10. The method of claim 9, wherein the detected border is detected by multi-slice computed tomography.

11. The method of claim 7, further comprising re-orienting a tip of the imaging catheter and capturing second image data.

12. The method of claim 11, further comprising performing an alignment using the intravascular image data and the second image data.

13. The method of claim 1, further comprising advancing the imaging catheter and obtaining a series of angiographic images.

14. The method of claim 1, further comprising measuring a distance that the imaging catheter advances using a caliper.

15. The method of claim 1, further comprising doing an IVUS pullback to obtain a 3D vessel wall.

16. The method of claim 1, further comprising sensing a position of the imaging catheter by triangulation.

17. The method of claim 16, wherein the triangulation uses low frequency ultrasound transducers.

18. The method of claim 16, wherein the triangulation uses electromagnets.

19. The method of claim 16, wherein the triangulation uses light sources and a light sensor on the catheter.
